(19) United States
(12) Patent Application Publication
(10) Pub. No.: US 2022/0265671 A1
(43) Pub. Date: Aug. 25, 2022

(54) METHOD FOR TREATING STROKE BY USING TRICYCLIC DERIVATIVE

(71) Applicant: Jeil Pharmaceutical Co., Ltd., Seocho-gu, Seoul (KR)

(72) Inventors: Joonwoo NAM, Seongnam-si, Gyeonggi-do (KR); In-Hae YE, Anyang-si, Gyeonggi-do (KR); Yongwoo LEE, Goyang-si, Gyeonggi-do (KR); Jongwoo KIM, Dobong-gu, Seoul (KR); Dongho LEE, Yongin-si, Gyeonggi-do (KR); Hyoeun JANG, Yongin-si, Gyeonggi-do (KR)

(21) Appl. No.: 17/630,700

(22) PCT Filed: Jul. 29, 2019
(86) PCT No.: PCT/KR2019/009444

§ 371 Date: Jan. 27, 2022

(51) Int. Cl. A61K 31/5377; A61K 9/19; A61K 47/02; A61K 47/26; A61P 9/10

(52) U.S. Cl. CPC A61K 31/5377; A61K 9/19; A61P 9/10; A61K 47/26; A61K 47/02

(57) ABSTRACT

A method for treating stroke by using a tricyclic derivative is described. More specifically, a dose and a regimen of administrating a tricyclic derivative that can exhibit optimal efficacy and effect as a therapeutic agent for use in treating a stroke patient are provided. The treatment method has the advantage of exhibiting optimal efficacy and effects while safely administering a novel tricyclic derivative.

### TECHNICAL FIELD

The present invention relates to a method for treating stroke by using a tricyclic derivative. More specifically, the present invention relates to a dose and a regimen of administrating a tricyclic derivative, according to the present invention, that can exhibit optimal efficacy and effect as a therapeutic agent for use in treating a stroke patient.

### BACKGROUND ART

Stroke is a disease with a high mortality rate because brain damage progresses within a few hours after the onset of the disease, and even though a stroke patient survives, stroke causes lifelong physical and mental disabilities such as quadriplegia, speech impairment, memory impairment, and mental impairment, so that stroke is a disease that causes a great burden both socially and economically.

Stroke is roughly divided into ischemic stroke in which a blockage of blood vessels causes necrosis of brain tissue, and hemorrhagic stroke, which is caused by ruptured blood vessels, and ischemic stroke accounts for about 80%.

In the case of ischemic stroke, a thrombolytic agent such as tissue plasminogen activator (tPA) or a thrombectomy are the only treatment method. Boehringer Ingelheim's Actilyse®, the only therapeutic agent currently approved for the treatment of stroke, normalizes blood flow and prevents brain damage by dissolving blood clots that block blood vessels when intravenously administered to an ischemic stroke patient within 4.5 hours of the onset of the disease. However, Actilyse® is effective only when administered within 4.5 hours of the onset of the disease, and when the drug is administered after 4.5 hours, the use of the drug is limited because the drug has a limitation of increasing side effects such as cerebral hemorrhage and death. Further, the effect is limited in patients with macrovascular occlusion. Recently, a stent-retriever thrombectomy was introduced to treat patients with macrovascular occlusion using a stent retriever with an improved reperfusion rate and speed, and five major clinical trials demonstrated that stent-retriever thrombectomy after tPA treatment significantly improved the prognosis of a patient compared to tPA therapy alone. However, 30 to 67% of patients with macrovascular occlusion who have undergone tPA treatment and stent-retriever thrombectomy treatment are unable to carry out independent daily life, and 12 to 30% of them have a very poor prognosis such as being bedridden or dying. Therefore, there is a need for the development of a drug capable of minimizing cell death and neuropathy by neuroprotective action as well as rapid reperfusion in order to further improve the prognosis of a stroke patient.

Most of the clinical drugs that have been attempted to be developed to date have tried to show a therapeutic effect by the mode of action which blocks cell apoptosis, but it was difficult to show a clinically large effect because brain damage due to cell death mainly occurs within the first 10 hours after the onset of stroke. Edaravone, a stroke treatment agent developed by Mitsubishi-Tanabe Pharma Corporation, Japan, is currently sold as a therapeutic agent for stroke only in Japan and China due to toxicity problems. In addition, a therapeutic agent for stroke Cerovive (NXY-059) developed by AstraZeneca also failed to prove its efficacy in Clinical Test—Phase III, so the development of the new drug was stopped.

Activation of poly(ADP-ribose) polymerase (PARP) by DNA damage in cerebral ischemia acts on cell death due to seizures, head damage and neurodegenerative diseases. Inhibition of PARP not only inhibits cell apoptosis, but also directly blocks necrosis of brain cells due to ATP energy depletion, and thus is likely to be developed as a therapeutic agent which protects cranial nerves. It was recently confirmed that a tricyclic derivative compound 10-ethoxy-8-(morpholinomethyl)-1,2,3,4-tetrahydrobenzo[h][1,6]naphthyridin-5(6H)-one, which is a ARP-1 inhibitor disclosed in Koran Patent No. 0968175, is effective in reducing cerebral infarction volume in a tMCAO animal model (Molecular Neurobiology 55(9859), January 2018).

### RELATED ART DOCUMENTS

### Patent Document

Korean Patent No. 0968175

### Non-Patent Document

Molecular Neurobiology 55(9859), January 2018

### DISCLOSURE

### Technical Problem

In order to solve the aforementioned problems, the present invention provides a pharmaceutical composition comprising a tricyclic derivative according to the present invention, a treatment method using the same, and a kit including the tricyclic derivative according to the present invention.

### Technical Solution

A “tricyclic derivative” used as an active ingredient of a pharmaceutical composition or preparation according to the present invention includes 10-ethoxy-8-(morpholinomethyl)-1,2,3,4-tetrahydrobenzo[h][1,6]naphthyridin-5(6H)-one, a pharmaceutically acceptable salt thereof, a hydrate thereof, a salt hydrate thereof, or a solvate thereof.

A pharmaceutically acceptable salt of the “tricyclic derivative” includes hydrochloric acid, benzenesulfonic acid, maleic acid, dimethanesulfonic acid, bis[(7,7-dimethyl-2-oxobicyclo[2,2,1]heptan-1-yl)methanesulfonic acid], tartaric acid, 2,6-dioxo-1,2,3,6-tetrahydropyrimidine-4-carboxylic acid, adipic acid, dinitric acid, fumaric acid, (S)-2-aminosuccinic acid, 2-hydroxypropane-1,2,3-tricarboxylic acid, cyclohexylsulphamic acid, sulfuric acid, succinic acid, formic acid, glutamic acid, diphosphoric acid, or the like. Furthermore, the tricyclic derivative may be present in the form of a hydrate or a salt hydrate, or a solvate. For example, an active ingredient in the pharmaceutical composition according to the present invention can be present in the form of 10-ethoxy-8-(morpholinomethyl)-1,2,3,4-tetrahydrobenzo[h][1,6]naphthyridin-5(6H)-one dihydrochloride or 10-ethoxy-8-(morpholinomethyl)-1,2,3,4-tetrahydrobenzo[h][1,6]naphthyridin-5(6H)-one dihydrochloride dihydrate.

A pharmaceutically acceptable carrier includes a sterile injectable solution, a sterile aqueous solution for the instant production of a dispersion, a dispersion or a sterile powder. A sterile aqueous solution, a dispersion, or ingredients (agents) which can be additionally included for a pharmaceutically active material are known in the art. Except for a case where any typical media or additional ingredients (agents) cannot be compatible with the tricyclic derivative of the present invention, the use thereof is contemplated in the pharmaceutical composition of the present invention. A pharmaceutically acceptable salt includes any suitable salts, dispersion media, coatings, antibacterial and antifungal agents, isotonic agents, antioxidants, absorption retardants, and the like, which are physiologically compatible with the tricyclic derivative of the present invention. Examples of suitable aqueous and non-aqueous carriers which can be used for the pharmaceutical composition of the present invention include distilled water, saline, phosphate buffered saline, ethanol, dextrose, polyols (for example, glycerol, propylene glycol, polyethylene glycol, and the like), and a suitable mixture thereof, a vegetable oil, such as olive oil, corn oil, peanut oil, cottonseed oil, and sesame oil, a carboxymethyl cellulose colloidal solution, tragacanth gum and an injectable organic ester, such as ethyl oleate, and/or various buffers. Other carriers are well known in the pharmaceutical field.

“Treatment” is defined as the application or administration of a tricyclic derivative of the present invention to a subject who has or is at risk of having a stroke, has stroke symptoms or is predisposed to develop a stroke, and herein, the purpose thereof is to cure, treat, mitigate, alleviate, modify, eliminate, ameliorate, improve, or influence stroke, the symptoms of stroke, or the causes that cause stroke. “Treatment” is also intended to apply or administer a pharmaceutical composition comprising a tricyclic derivative of the present invention to a subject, and herein, the purpose thereof is to cure, treat, mitigate, alleviate, modify, eliminate, ameliorate, improve, or influence a disease, the symptoms of the disease, or the causes that cause the disease.

The pharmaceutical composition applied in the present invention preferably comprises a “therapeutically effective amount” of the tricyclic derivative according to the present invention.

A “therapeutically effective amount” or “effective amount” of the composition for stroke means an amount of the composition which delays, reduces, alleviates, ameliorates, stabilizes, suppresses and/or reverses one or more stroke-related symptoms (clinical symptoms, biochemical symptoms and the like, for example, physical disabilities such as brain cell necrosis or cell apoptosis due to reperfusion injury, the resulting increase in cerebral infarction volume, and limb paralysis and facial muscle paralysis, mental disabilities such as speech impairment, memory impairment, a decrease in cognitive ability) compared to the absence of the composition in an embodiment. This includes the dosage and duration required to achieve a desired therapeutic result. The term “delay” of symptoms refers to an increase in the period between exposure to the tricyclic derivative according to the present invention and the onset of one or more symptoms described herein. The term “elimination” of symptoms refers to a reduction in one or more symptoms described herein of 40, 50, 60, 70, 80, 90, or even 100%. The therapeutically effective amount also includes an amount where a therapeutically beneficial effect is greater than any toxic or detrimental effect of the composition.

“Administration” refers to the administration of a material to achieve a therapeutic purpose. In the present invention, “administration” includes intravenous administration. Administration may be performed once or more to achieve a desired therapeutic effect.

q“Subject” includes all human or non-human animals. The term “non-human animal” includes a vertebrate such as a non-human primate, a cow, a pig, a horse, a sheep, a dog, a cat, a rabbit and a white ferret, a rodent such as a mouse, a rat and a guinea pig, a bird species such as a chicken, an amphibian, and a reptile, but is not limited thereto. In a preferred embodiment, the subject is a mammal, such as a non-human primate, a cow, a pig, a horse, a sheep, a dog, a cat, a rabbit, a white ferret or a rodent. In a more preferred embodiment, the subject is a human. The terms “subject”, “patient” and “individual” are used interchangeably herein.

“Ischemia” refers to a condition in which oxygen is deficient due to insufficient supply of blood because the blood vessels that supply blood are narrowed or constricted, or normal angiogenesis is not sufficiently performed.

“Reperfusion” refers to the re-flow of blood into blood vessels to prevent tissue damage caused by ischemia.

“Kit” refers to a packaged product including ingredients for administering the tricyclic derivative of the present invention for the treatment of stroke. The corresponding kit includes a container or box which holds the ingredients of the kit. The kit also includes instructions for administering the tricyclic derivative of the present invention.

The present invention provides

a pharmaceutical composition for use in treating stroke, comprising a therapeutically effective amount of 10-ethoxy-8-(morpholinomethyl)-1,2,3,4-tetrahydrobenzo[h][1,6]naphthyridin-5(6H)-one, a pharmaceutically acceptable salt thereof, hydrate thereof, salt hydrate thereof or solvate thereof as an active ingredient and a pharmaceutically acceptable carrier,

wherein a first dose of the pharmaceutical composition comprising 8 to 20 wt % of the active ingredient based on a single dose of the active ingredient is intravenously administered to a subject at 5 to 15 mg/min based on the active ingredient, and

a second dose of the pharmaceutical composition comprising the remaining dose of the active ingredient (based on the active ingredient) is intravenously administered to the subject for 20 to 26 hours.

Further, the present invention provides a method for treating stroke, the method including: administering, to a subject in need thereof, a pharmaceutic composition comprising a therapeutically effective amount of 10-ethoxy-8-(morpholinomethyl)-1,2,3,4-tetrahydrobenzo[h][1,6]naphthyridin-5(6H)-one, a pharmaceutically acceptable salt thereof, hydrate thereof, salt hydrate thereof or solvate thereof as an active ingredient as an active ingredient and a pharmaceutically acceptable carrier,

wherein the pharmaceutical composition is divided into a first dose of the pharmaceutical composition and a second dose of the pharmaceutical composition and administered,

a first dose of the pharmaceutical composition comprising 8 to 20 wt % of the active ingredient based on a single dose of the active ingredient is intravenously administered to a subject at 5 to 15 mg/min based on the active ingredient, and

a second dose of the pharmaceutical composition comprising the remaining dose of the active ingredient (based on the active ingredient) is intravenously administered to the subject for 20 to 26 hours.

According to the following examples, it is preferred that the pharmaceutical composition according to the present invention be administered in divided doses in the case of a single administration. The method of administering in divided doses is preferred because a first dose of the pharmaceutical composition is rapidly administered based on a single dose of the active ingredient, and thus the tricyclic derivative according to the invention will rapidly achieve a desired blood concentration. The remaining dose of the pharmaceutical composition other than the first dose of the pharmaceutical composition, that is, the second dose of the pharmaceutical composition, will then be slowly administered to the subject, and thus allows the tricyclic derivative to be able to maintain a desired blood concentration at a predetermined level. For stroke patients, rapid administration of the tricyclic derivative according to the present invention and maintenance of the blood concentration at a predetermined level are very important for obtaining an appropriate therapeutic effect.

In this case, the first dose of the pharmaceutical composition to be first administered is determined as an amount of the pharmaceutical composition comprising 8 to 20 wt %, such as 10 to 20 wt %, 12 to 20 wt %, and 15 to 18 wt % of the active ingredient, based on the single dose of the active ingredient.

The second dose of the pharmaceutical composition administered after the first dose of the pharmaceutical composition becomes an amount of the pharmaceutical composition remaining after the first dose of the pharmaceutical composition is administered. For example, when the first dose is 8 to 20 wt % of the active ingredient, the second dose becomes a pharmaceutical composition comprising 92 to 80 wt % of the active ingredient.

In the present invention, the first dose of the pharmaceutical composition comprising 8 to 20 wt % of the active ingredient based on the single dose of the active ingredient is intravenously administered to a subject at 5 to 15 mg/min based on the active ingredient. As described above, the first dose of the pharmaceutical composition is administered such that 10-ethoxy-8-(morpholinomethyl)-1,2,3,4-tetrahydrobenzo[h][1,6]naphthyridin-5(6H)-one rapidly reaches a therapeutically effective concentration in the body. The first dose of the pharmaceutical composition may be administered at a rate of 5 to 15 mg/min, for example, 7 to 13 mg/min, 7 to 11 mg/min, such as 8 to 11 mg/min based on the active ingredient.

Furthermore, following the administration of the first dose of the pharmaceutical composition, the second dose of the pharmaceutical composition comprising the remaining dose of the active ingredient is intravenously administered to a subject based on the active ingredient for 20 to 26 hours. The administration rate of the second dose of the pharmaceutical composition may be appropriately adjusted such that 10-ethoxy-8-(morpholinomethyl)-1,2,3,4-tetrahydrobenzo[h][1,6]naphthyridin-5(6H)-one maintains the blood concentration at a suitable level in the body. The second dose of the pharmaceutical composition may be administered by being appropriately adjusted within a range of, for example, 20 to 26 hours, such as 21±1 hours, 22±1, 23±1, 24±1, 25±1 hours (based on the active ingredient).

The first dose may be administered as a bolus, and the second dose may be administered by IV infusion, but are not limited thereto. The first dose and the second dose may be contained in one container or in separate containers, respectively. For example, the first dose and the second dose may be contained in one container, and the pharmaceutical composition of the present invention may be continuously injected into a vein at an administration rate of the first dose and then at an administration rate of the second dose using an infusion pump. Various infusion pumps are currently commercially available, and it is possible to adjust the time and rate of administration such that the pharmaceutical composition is intravenously injected in the form of IV infusion after bolus administration.

The single dose of the tricyclic derivative according to the present invention may be appropriately selected in consideration of the state and severity of stroke symptoms at the time of treatment, stroke treatment history, such as the presence or absence of tPA administration, and in consideration of the age, body weight, gender, health condition, medication being administered, and the like of the subject as a whole.

In the present invention, the single dose of the active ingredient may be 700 to 2000 mg.

In an embodiment of the present invention, the single dose of the active ingredient may be 700 to 1100 mg. As previously described, the single dose of the active ingredient may be divided into a first dose and a second dose and administered.

For example, when the active ingredient is 10-ethoxy-8-(morpholinomethyl)-1,2,3,4-tetrahydrobenzo[h][1,6]naphthyridin-5(6H)-one dihydrochloride, the single dose of the active ingredient may be 900 mg. Although not limited thereto, in this case, the first dose may be set to 150 mg for a single dose of 900 mg, and the second dose may be set to 750 mg, which is the dose remaining after the first dose. The first dose of the pharmaceutical composition may be intravenously administered to the subject at 5 to 15 mg/min based on the active ingredient, and the second dose of the pharmaceutical composition may be intravenously administered to the subject based on the active ingredient for 20 to 26 hours.

In another embodiment of the present invention, the single dose of the active ingredient may be 1600 to 2000 mg. As previously described, the single dose of the active ingredient may be divided into a first dose and a second dose and administered.

For example, when the active ingredient is 10-ethoxy-8-(morpholinomethyl)-1,2,3,4-tetrahydrobenzo[h][1,6]naphthyridin-5(6H)-one dihydrochloride, the single dose of the active ingredient may be 1800 mg. Although not limited thereto, in this case, the first dose may be set to 300 mg for a single dose of 1800 mg, and the second dose may be set to 1500 mg, which is the dose remaining after the first dose. The first dose of the pharmaceutical composition may be intravenously administered to the subject at 5 to 15 mg/min based on the active ingredient, and the second dose of the pharmaceutical composition may be intravenously administered to the subject based on the active ingredient for 20 to 26 hours.

Although not limited thereto, the first dose of the pharmaceutical composition and the second dose of the pharmaceutical composition may be sequentially and continuously administered to the subject. Immediately after the first dose of the pharmaceutical composition is completely administered, the second dose of the pharmaceutical composition may be immediately administered to the subject.

The subject to which the pharmaceutical composition according to the present invention is administered is not particularly limited as long as it is a stroke patient. Since tPA is a thrombolytic agent, tPA is administered to a patient in need of reperfusion, whereas since 10-ethoxy-8-(morpholinomethyl)-1,2,3,4-tetrahydrobenzo[h][1,6]naphthyridin-5(6H)-one used as an active ingredient in the present invention has an effect of protecting the cranial nerves by not only inhibiting cell apoptosis due to ischemic damage but also directly blocking necrosis of brain cells due to ATP energy depletion, the subject to which the pharmaceutical composition according to the present invention is administered includes all of a subject who requires reperfusion due to the onset of a stroke symptom or a subject who has undergone reperfusion after the onset of a stroke symptom. For example, both a patient before and after administration of tPA as a stroke patient and a patient before and after undergoing thrombectomy may be a subject to which the pharmaceutical composition according to the present invention is administered.

It is preferred that a subject who is ischemic due to a stroke undergo reperfusion again within a rapid period of time.

Although not limited thereto, in the present invention, it is preferred that the reperfusion is performed within 24 hours, such as within 20 hours, within 16 hours, within 12 hours, within 10 hours, within 8 hours, within 6 hours, and within 4.5 hours after the onset of a stroke symptom.

Meanwhile, the blood concentration of the active ingredient after administration of the pharmaceutical composition according to the present invention may be 1000 μg/L or more for 24 hours. This is desirable for achieving the therapeutic effect of the pharmaceutical composition according to the present invention.

The pharmaceutical composition according to the present invention may be administered in combination with tPA. The pharmaceutical composition of the present invention may be administered to a subject who has already undergone reperfusion by, for example, tPA, or may be simultaneously administered with tPA.

In the present invention, the subject may be a mammal, preferably a human.

In an embodiment of the present invention, the subject includes a human having one or more of the following characteristics.

a) a person confirmed to have acute cerebral artery occlusion in the intracranial internal carotid artery (IICA) or the middle cerebral artery (MCA) in CT angiography, MR angiography, or TFCA;

b) a person with a score of 6 to 30 points in the Korean-National Institutes of Health Stroke Scale (K-NIHSS) before endovascular recanalization therapy (ERT);

c) a person with modified thrombolysis in cerebral infarction (mTICI) who has been reperfused within 24 hours, 10 hours or 6 hours after the onset of symptoms;

d) a person who has already been confirmed to have reperfusion before thrombectomy due to a venous tPA effect when angiography was performed for thrombectomy after intravenous tPA treatment;

e) a person with a pre-mRS of 0 to 1 before the onset of the disease.

Although not limited thereto, in an embodiment of the present invention, the Korean version of a modified Rankin Scale (K-mRS) measured at a time point of 90 days after administration of the pharmaceutical composition according to the present invention may be 2.5 or less, preferably 2 or less. When the K-mRS is 0 to 2, the patient is evaluated as being capable of independent daily activities. Administration of the pharmaceutical composition according to the present invention exhibits a very excellent stroke therapeutic effect that allows the patient to reach a K-mRS of 2.5 or less within 90 days.

Meanwhile, the pharmaceutical composition according to the present invention may comprises additional pharmaceutically acceptable additives such as a pH adjuster, a stabilizer, and an isotonic agent in addition to an active ingredient and a pharmaceutically acceptable carrier.

In an embodiment of the present invention, the pharmaceutical composition may include a pH adjuster. The pH adjuster refers to a neutralizing material that minimizes changes in pH due to acids or alkalis. Examples of the pH adjuster include, but are not limited to, sodium carbonate, sodium hydrogen carbonate, potassium hydrogen carbonate, sodium hydroxide, sodium citrate, potassium diphosphate, potassium triphosphate, potassium hydroxide, potassium carbonate, potassium phosphate or a mixture thereof.

The pharmaceutical composition according to the present invention may also additionally comprise a sugar or a derivative thereof. The sugar or the derivative thereof may serve as a stabilizer or an isotonic agent in a pharmaceutical composition. The sugar may include monosaccharides, disaccharides, oligosaccharides, polysaccharides or mixtures of two or more thereof. Examples of monosaccharides include glucose, fructose, galactose, and the like, but are not limited thereto. Examples of disaccharides include sucrose, lactose, maltose, trehalose, and the like, but are not limited thereto. Examples of oligosaccharides include fructooligosaccharides, galactooligosaccharides, mannan oligosaccharides, and the like, but are not limited thereto. Examples of polysaccharides include starch, glycogen, cellulose, chitin, pectin, and the like, but are not limited thereto.

The derivative of sugar may include sugar alcohols, sugar acids, or a mixture thereof. Examples of sugar alcohols include glycerol, erythritol, threitol, arabitol, xylitol, ribitol, mannitol, sorbitol, galacticol, fusitol, iditol, inositol, volemitol, isomalt, maltitol, lactitol, maltotritol, maltotetraitol, polyglycitol, and the like, but are not limited thereto. Examples of sugar acids include (glyceric acid, and the like), ulosonic acids (neuraminic acid, and the like), uronic acids (glucuronic acid, and the like), aldaric acids (tartaric acid, and the like), and the like, but are not limited thereto.

In an embodiment of the present invention, mannitol, sorbitol, erythritol or a mixture of two or more thereof may be included as a sugar or a derivative thereof. The pharmaceutical composition according to the present invention may include D-mannitol, but is not limited thereto.

As an additional additive, an isotonic agent may also be included, and for example, sodium chloride, glucose, boric acid, glycerin, potassium chloride, corn syrup, and the like may be used.

Although not limited thereto, the active ingredient according to the present invention is formulated in the form of a lyophilized powder or cake, and thus may be used by being dissolved in typical water for injection, such as physiological saline, if necessary. An additional additive such as a pH adjuster and/or a stabilizer may also be used by being dissolved in a suitable amount of physiological saline. Otherwise, pharmaceutically acceptable additives such as a pH adjuster and/or a stabilizer may be provided in the form of solutions in which they are dissolved.

Although not limited thereto, a solution in which the active ingredient is dissolved and a solution in which the pH adjuster and/or stabilizer are/is dissolved may be sequentially infused into an injection bag including typical water for injection, such as physiological saline to obtain a pharmaceutical composition according to the present invention in the form of a liquid preparation.

In an embodiment of the present invention, the pharmaceutical composition of the present invention may have a pH of 7 or less, preferably a pH of 2.5 to 7, such as a pH of 3 to 7, a pH of 3.5 to 6.5, a pH of 4 to 6, a pH of 4.5 to 6, and a pH of 5 to 6. This is a range suitable for preventing the precipitation of the active ingredient and injecting the pharmaceutical composition into a subject.

In another aspect, the present invention provides a kit including a preparation including a therapeutically effective amount of 10-ethoxy-8-(morpholinomethyl)-1,2,3,4-tetrahydrobenzo[h][1,6]naphthyridin-5(6H)-one, a pharmaceutically acceptable salt thereof, hydrate thereof, salt hydrate thereof or solvate thereof as an active ingredient and instructions which instruct a use method of administering the preparation to a subject.

The preparation may have a formulation such as a liquid preparation, a lyophilized powder or cake by a method known to those skilled in the art. When the active ingredient is formulated as a lyophilized powder or cake, it may be used by reconstitution into a liquid preparation at an appropriate concentration by being mixed with water for injection, if necessary, immediately before administration to a subject.

In an embodiment, the preparation may be a lyophilized powder or cake. In this case, the kit may further include a pharmaceutically acceptable carrier for reconstitution of a preparation in the form of a lyophilized powder or cake into a liquid preparation. In addition, the kit may further include a pharmaceutically acceptable additive such as a pH adjuster and/or a stabilizer. The additives are as described above.

In the kit according to the present invention, the instructions further include a label or imprint indicating that contents included in the kit may be used for the treatment of stroke by administering the tricyclic derivative according to the present invention to a subject for the treatment of stroke in the subject.

In an embodiment of the present invention, the instructions may mean instructions which instruct a use method of administering the preparation to a subject, the method including reconstituting the preparation into a liquid preparation by dissolving the preparation in a pharmaceutically acceptable carrier, then preparing a pharmaceutical composition for administration by mixing the liquid preparation with water for injection, intravenously administering a first dose of the pharmaceutical composition comprising 8 to 20 wt % of the active ingredient based on a single dose of the active ingredient at 5 to 15 mg/min to a subject, and intravenously administering a second dose of the pharmaceutical composition comprising the remaining dose of the active ingredient based on the active ingredient to the subject for 20 to 26 hours.

The instructions may include a description on a process of obtaining the pharmaceutical composition according to the present invention in the form of a liquid preparation by infusing a solution in which the preparation is dissolved in a pharmaceutically acceptable carrier into an injection bag containing typical water for injection (for example, physiological saline) and separately infusing a solution in which a pharmaceutically acceptable additive such as a pH adjuster and/or a stabilizer is dissolved in a suitable amount of water for injection (for example, physiological saline) into the injection bag. Further, it is possible to include a description on a use method of administering the preparation to a subject, the method including intravenously administering a first dose of the pharmaceutical composition comprising 8 to 20 wt %/o of the active ingredient based on a single dose of the active ingredient at 5 to 15 mg/min to a subject, and intravenously administering a second dose of the pharmaceutical composition comprising the remaining dose of the active ingredient (based on the active ingredient) to the subject for 20 to 26 hours.

Additionally, the kit according to the present invention may further include a means or vial, Teflon bag or infusion bag (typically used for infusion of a therapeutic agent) for administering a first dose of the pharmaceutical composition and a second dose of the pharmaceutical composition of the present invention to a subject. Here, the “means” includes a syringe, an injection needle, a cannula, a catheter, an infusion bag for intravenous administration, an intravenous vehicle, a light-shielding bag, a light-shielding line, a light-shielding tubing cover, and the like.

The pharmaceutical composition according to the present invention may be additionally mixed with water for injection and then administered using an infusion pump. Since the infusion pump enables IV infusion at different speeds, it is desirable to use the infusion pump to maintain an appropriate blood concentration of the active ingredient.

### Advantageous Effects

The pharmaceutical composition according to the present invention can be used for the protection of nerve cells in the brain regardless of reperfusion, and thus provides a very excellent therapeutic effect on stroke. Further, the method for administering the pharmaceutical composition according to the present invention provides an advantage of exhibiting optimal efficacy and effect while safely administering a novel tricyclic derivative.

### DESCRIPTION OF DRAWINGS

FIG. 1 shows the goodness of fit of a test drug population pharmacokinetics model.

FIG. 2 shows individual observations and predictions of concentrations of the test drug over time.

FIG. 3 shows a visual predictive test in the test drug population pharmacokinetics model.

FIG. 4 shows a simulation of the blood concentration of the test drug over time.

FIG. 5 shows a simulation of blood concentration over time when 900 mg of the test drug is administered by IV-infusing 750 mg of the test drug up to 24 hours after administration of a 150 mg bolus of the test drug for 15 minutes.

FIG. 6 shows a simulation of blood concentration over time when 1800 mg of the test drug is administered by IV-infusing 1500 mg of the test drug up to 24 hours after administration of a 300 mg bolus of the test drug for 30 minutes.

### MODES OF THE INVENTION

Hereinafter, the present invention will be described in detail with reference to Examples. The following Examples are only for exemplifying the present invention, and the scope of the present invention is not limited to the following Examples.

Medicine for Clinical Test

Test Drug
Main agent: a lyophilized cake or powder/vial of 300 mg of test drug (as 10-ethoxy-8-(morpholinomethyl)-1,2,3,4-tetrahydrobenzo[h][1,6]naphthyridin-5(6H)-one dihydrochloride)
Buffered solvent: 150 mg of di-mannitol, 90 mg of sodium hydrogen carbonate, a suitable amount of water for injection/a vial

Control Drug (Placebo)
Main agent: 0.2 mg of riboflavin sodium phosphate, a lyophilized cake or powder of 150 mg of D-mannitol/a vial

Buffered solvent: a suitable amount of water for injection/a vial

Preparation Method
Preparation of injection of 900 mg dose group:

\textcircled{1} Extract 18 ml of 0.9% sterile physiological saline for injection from 1.0 L of 0.9% sterile physiological saline for injection using a 20 ml-syringe and dissolve a test drug by infusing each of 6 ml of the saline into 3 main agent vials.

\textcircled{2} Take the whole amount (6 ml×3 vials, total 18 ml) of 0.9% sterile physiological saline in which 300 mg of the test drug is dissolved using a 20 ml-syringe and infuse the same into 1.0 L of the 0.9% sterile physiological saline for injection to a subject in \textcircled{1}.

\textcircled{3} Take a total of 18 ml of each 6 ml from 3 vials containing a clear buffered solution using a 20 ml-syringe and infuse the same into 1.0 L of the 0.9% sterile physiological saline for injection to a subject in \textcircled{2}.

\textcircled{4} Immediately pack 1.0 L of the 0.9% sterile saline for injection containing the completely prepared injection solution in a light-shielding envelope.

\textcircled{5} In principle, an injection for administration should be prepared and immediately used after preparation.

### Example 1. Phase 1 Clinical Test of Test Drug Single Dose Escalation

In order to evaluate the safety at the time of a single dose of a test drug, a phased clinical test was conducted as determined by the Data Safety Monitoring Board (DSMB) on 40 subjects (8 subjects per cohort: 6 subjects in test group and 2 subjects in placebo group) in 5 dose groups (35 mg, 75 mg, 150 mg, 300 mg, 600 mg) selected from healthy male volunteers in Korea.

Administration Method

A test drug or placebo was mixed with an infusion solution by intravenous injection therapy, and then administered using an infusion pump for 30 minutes (±5 minutes).

Evaluation of Adverse Reaction

Of the 40 test subjects who were administered the clinical test drug, a total of 6 test subjects developed 7 adverse reactions. There were no reported adverse reactions in the placebo-administered group. Of the 7 adverse reactions, 1 case of “dizziness” was a moderate symptom in the test subjects who were administered 600 mg, and all other adverse reactions corresponded to a mild symptom. The subjects were spontaneously recovered from all the adverse reactions without any sequelae. No significant adverse reactions occurred throughout the entire clinical test. Accordingly, it was determined that the drug tolerability of a single dose of the test drug was good in the test dose range.

### Example 2. Phase 1 Clinical Test of Test Drug Multi Dose Escalation

In order to evaluate the safety at the time of a repeated dose of a test drug, a phased clinical test was conducted as determined by the Data Safety Monitoring Board (DSMB) on 24 subjects (8 subjects per cohort: 6 subjects in test group and 2 subjects in placebo group) in 3 dose groups (repeated administration of 150 mg, 300 mg, and 450 mg seven times at intervals of 12 hours) selected from healthy male volunteers in Korea.

Administration Method

A test drug or placebo was mixed with an infusion solution by intravenous injection therapy, and then administered using an infusion pump for 60 minutes (±10 minutes), and the same was repeatedly administered seven times at intervals of 12 hours.

Evaluation of Adverse Reaction

Of the 24 test subjects who were administered the clinical test drug, a total of 7 test subjects developed 12 adverse reactions, and among them, 10 cases were confirmed to be adverse drug reactions. One case (one person) of an adverse reaction occurred in the placebo-administered group. All the adverse reactions were mild, the subjects were spontaneously recovered from all the adverse reactions without any sequelae, and no significant adverse reactions occurred throughout the entire clinical test. Accordingly, it was determined that the tolerability of about 150 to 450 mg of the test drug during the repeated administration at intervals of 12 hours was good.

### Example 3. Construction and Simulation of Population Pharmacokinetics Model

A pharmacokinetics model for administration of a test drug was established for integration with a quantitative pharmacokinetics model for simulating the efficacy and safety of test drugs, as well as simulating the pharmacokinetics of future doses and dosage regimens. The simulation was performed by constructing a pharmacokinetics model based on pharmacokinetics information and demographic information of a total of 30 test subjects who were administered 35 mg, 75 mg, 150 mg, 300 mg, and 600 mg among the test subjects who participated in the single dose clinical test of test drug phase 1. The analysis was performed by a non-linear mixed effect modeling method using NONMEM® (version 7.2; ICON Development Solutions, Ellicott City, Md., USA).

The population pharmacokinetics model was constructed in the order of structural model construction, covariate analysis, and model selection. As a first step, a basic structural model was constructed, and as a step of establishing a model capable of best explaining the change in drug concentration over time, the most pharmacologically and statistically suitable model was selected by performing a search in the order of a 1-compartment model, a 2-compartment model, and a 3-compartment model. As the next step, covariate analysis was performed, and the effects of age, height, body weight, serum creatine, BUN, albumin, AST, and ALT concentrations which are part of the demographic information on pharmacokinetics were quantitatively evaluated. Finally, model selection (internal validation) was performed, a simulation of the established model was performed, and this was evaluated by a visual predictive check (VPC), which is a schematic evaluation method.

Based on the constructed model, pharmacokinetics patterns for various doses/dosage regimens were simulated. Various doses were simulated from the case of bolus administration for 30 minutes to the cases of infusion administration for 1 hour, infusion administration for 2 hours, infusion administration for 12 hours, infusion administration for 24 hours, and the like. In addition, various doses were simulated from the cases of infusion for 12 hours after bolus administration for 15 minutes, infusion for 24 hours after bolus administration for 30 minutes, and the like.

Based on these simulation results, a dose/dosage regimen capable of maintaining a target blood concentration was investigated.

Construction of Population Pharmacokinetics Model

The pattern of time-blood concentration changes of the test drug is best explained by a 3-compartment model with 1st order elimination. No significant covariate was found in the covariate analysis utilizing age, height, body weight, serum creatine, BUN, albumin, AST, and ALT concentrations. The results of each pharmacokinetics parameter prediction analysis are the same as in Table 1.

TABLE 1
Pharmacokinetics parameter prediction and standard
error in test drug population pharmacokinetics model

| | | Relative |
|---|---|---|
| | Predicted | standard |
| Parameter | value | error (%) |
| Structural model | | |
| Clearance of central | 32.0 | 8.3 |
| compartment (CL, L/h) | | |
| Volume of central | 15.2 | 10.2 |
| compartment (V1, L) | | |
| 1^{st} Clearance of peripheral | 162 | 10.9 |
| compartment (Q1, L/h) | | |
| 1^{st} Volume of peripheral | 58.5 | 4.0 |
| compartment (V2, L) | | |
| 2^{nd} Clearance of peripheral | 2.66 | 16.9 |
| compartment (Q2, L/h) | | |
| 2^{nd} Volume of peripheral | 8.81 | 18.8 |
| compartment (V3, L) | | |
| Interindividual model | | |
| CL (%) | 43.8 | 19.4 |
| V1 (%) | 52.4 | 25.2 |
| Q1 (%) | 41.7 | 33.0 |
| V2 (%) | 20.2 | 24.3 |
| Q2 (%) | 28.1 | 212.7 |
| V3 (%) | 64.1 | 61.8 |
| Residual error | | |
| Additive residual error (μg/L) | 0.517 | 56.9 |
| Proportional residual error (%) | 0.108 | 8.2 |

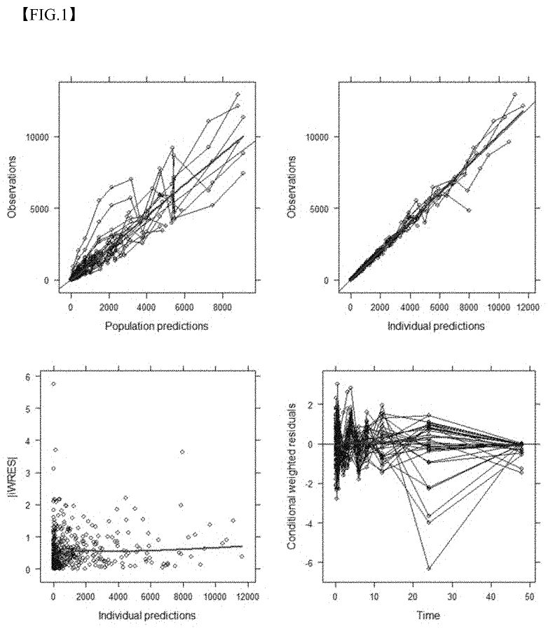

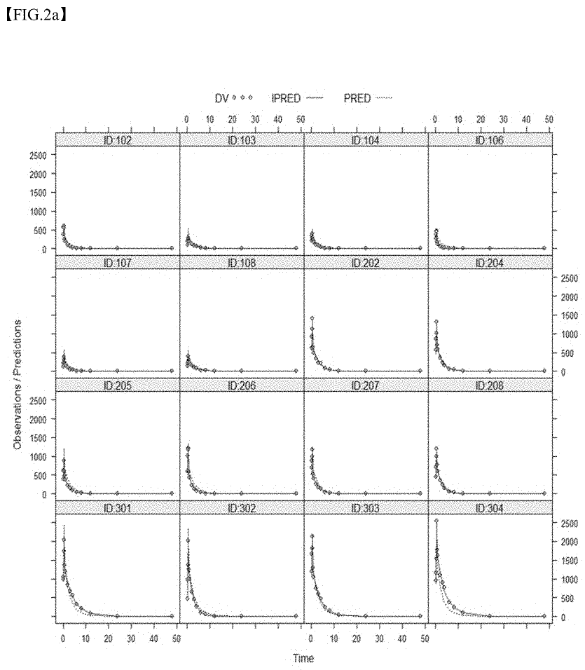

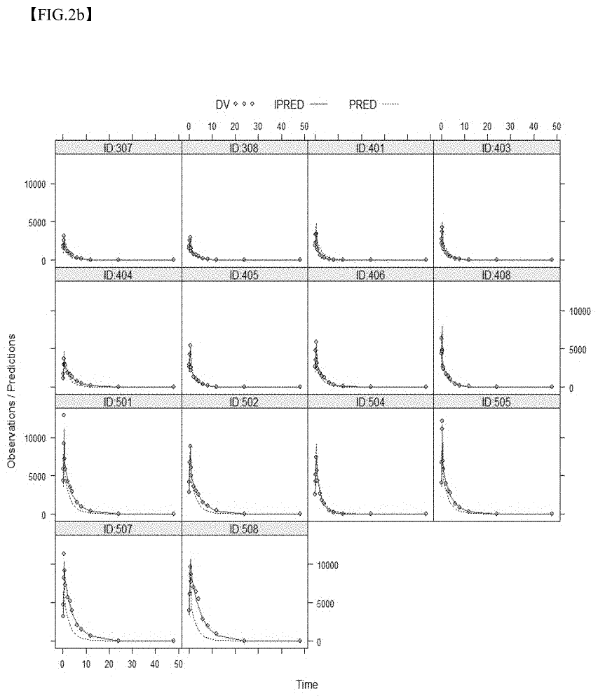

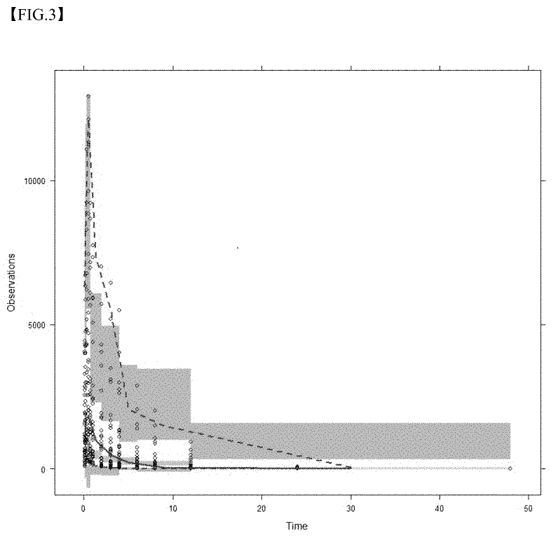

The population pharmacokinetics analysis results were very consistent with the observed data, which can be confirmed by the goodness-of-fit graph in FIG. 1 and the individually measured value and predicted value graphs in FIG. 2. In addition, as a result of the visual predictive check (VPC), most of the data was present within 5% to 95% (FIG. 3), and through this, it could be confirmed that the population pharmacokinetics model is a model appropriate for predicting the pharmacokinetics of the test drug.

Simulation

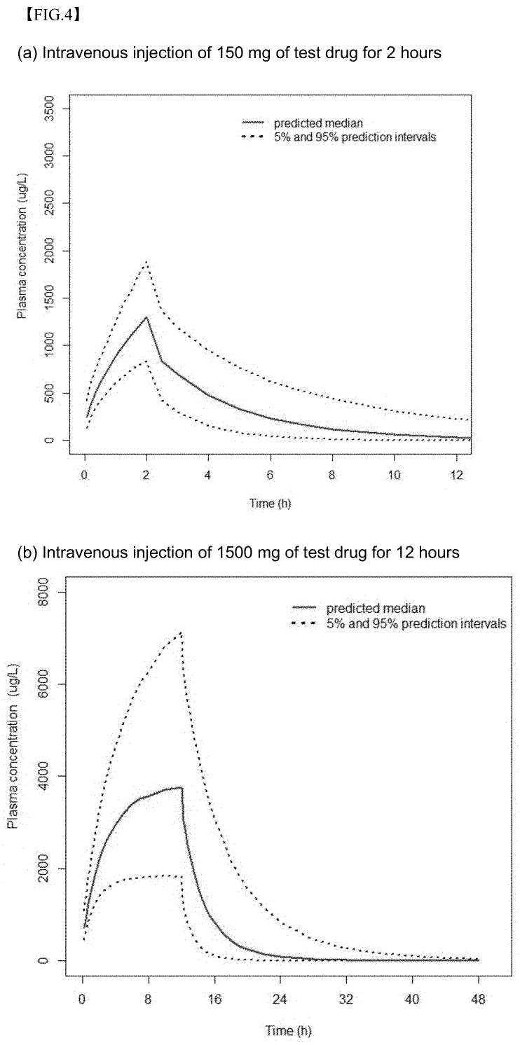

Pharmacokinetics patterns in various administration doses/dosage regimens were understood by simulating the cases of administering a dose from 150 mg to 1500 mg of the test drug by bolus administration for 30 minutes, infusion administration for 1 hour, infusion administration for 2 hours, infusion administration for 12 hours, and infusion administration for 24 hours. Some of the simulation results were the same as in FIG. 4.

Since ischemic stroke, which is an indication for the test drug, is a disease which requires acute therapy, it is clinically important to reach a blood/intracellular concentration which is equal to or more than the effective concentration at the initial stage. Therefore, it was determined that it is necessary to rapidly increase the blood drug concentration by bolus administration at the initial stage. The test drug is a PARP inhibitor, but to date, the changes (reversibility, and the like) caused by the PARP inhibitor in stroke patients have not been clearly elucidated. However, it is known that brain cell necrosis or cell apoptosis occurs intensively within 24 hours due to reperfusion injury which is inevitably accompanied by thrombectomy. Therefore, it was determined that it is necessary to continuously maintain the intracellular drug concentration which is equal to or more than a certain concentration by maintaining the blood drug concentration which is equal to or more than a predetermined concentration, thereby enabling the PARP inhibitor according to the present invention to continuously act. Therefore, it was determined that it is necessary to maintain the drug concentration which is equal to or more than the target blood concentration for up to 24 hours by infusion after bolus.

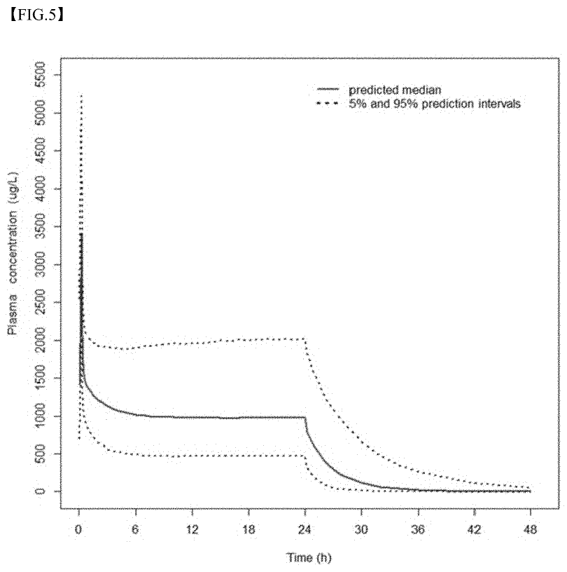

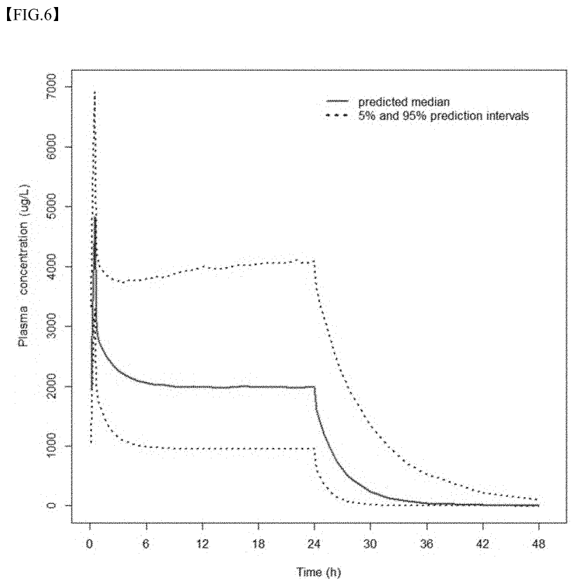

Thus, as the next step, simulations on a bolus+infusion dosage regimen, which can quickly reach the initial target blood concentration or more and maintain the target blood concentration for up to 24 hours, were performed at various doses. Among them, for a dosage of continuous infusion of 750 mg of the test drug for up to 24 hours after bolus administration of 150 mg of the test drug for 15 minutes, the maximum blood drug concentration was estimated to be about 3500 μg/L on average, and the maintenance blood drug concentration was estimated to be about 1000 μg/L, and for a dosage regimen of continuous infusion of 1500 mg of the test drug for up to 24 hours after bolus administration of 300 mg of the test drug for 30 minutes, the maximum blood drug concentration was estimated to be about 5000 μg/L on average, and the maintenance blood drug concentration was estimated to be about 2000 μg/L. The predicted blood drug concentration-time pattern of the two dosages is the same as in FIGS. 5 and 6, and the predicted blood drug concentration over time is the same as in Table 2.

TABLE 2
Simulation of blood concentration of test drug

| | Test drug blood concentration simulation (μg/L)* | | | |
|---|---|---|---|---|
| Time | 150 mg bolus + | | 300 mg bolus + | |
| (h) | 750 mg infusion | | 1500 mg infusion | |
| 0 | 0 | | 0 | |
| 0.25 | 3419 | (2221, 5224) | 4832 | (3295, 6916) |
| 1 | 1367 | (821, 2035) | 2734 | (1692, 4018) |
| 2 | 1215 | (654, 1933) | 2446 | (1327, 3837) |
| 4 | 1070 | (524, 1893) | 2159 | (1057, 3763) |
| 6 | 1015 | (486, 1905) | 2055 | (982, 3795) |
| 8 | 990 | (476, 1932) | 2013 | (961, 3819) |
| 12 | 981 | (468, 1962) | 1982 | (955, 3993) |
| 24 | 977 | (469, 2018) | 1978 | (946, 4099) |
| 36 | 23 | (1, 260) | 47 | (2, 523) |
| 48 | 2 | (0, 50) | 4 | (0, 101) |

*Median (minimum, maximum)

Evaluation

In consideration of the simulation results and pharmacokinetic-drug history characteristics of the drug, it was determined that the two types of doses/dosage regimens are appropriate.

### Example 4. Evaluation of Safety and Therapeutic Efficacy During Administration of Test Drug in Stroke Patients

A multi-organ, randomly allocated, double-blind, placebo-controlled, and initial phase II clinical test was conducted on patients with acute ischemic stroke to explore and evaluate the efficacy and safety of the study drug, respectively. This clinical test includes a total of three cohorts, and test subject registration starts from Cohort 1, and when the safety is secured, Cohort 2 is sequentially registered. The registration of Cohort 2 proceeds when the safety is secured after evaluation by the Data Safety Monitoring Board (DSMB) at the time point when all the test subjects of Cohort 1 completed Visit 5 (Day 29). Cohorts 1 and 2 are for confirming the safety and efficacy of the test drug, and based on this, the dose and dosage regimen, and the like of Cohort 3 are determined.

This clinical test is conducted on patients with modified thrombolysis in cerebral infarction (mTICI) 2b or 3 grade reperfusion in angiography after endovascular recanalization therapy (ERT) among patients with severe ischemic stroke in moderate cases confirmed to have undergone acute cerebral artery occlusion of the anterior circulation system. Intravenous tissue plasminogen activator (IV tPA) intravenous administration is permitted when the stroke is indicated before ERT. Furthermore, when angiography is performed for ERT after intravenous tPA administration, patients who have already undergone mTICI 2b-3 reperfusion by a venous tPA effect alone before ERT also become a subject of the clinical test.

Test subjects could be registered in this clinical test only when they met all of the following criteria.

Screening Selection Criteria
Men and women equal to or more than 19 years of age with acute ischemic stroke
a person confirmed to have acute cerebral artery occlusion in the intracranial internal carotid artery (IICA) or the anterior circulatory region of the middle cerebral artery (MCA) MI segment in CT angiography, MR angiography, or TFCA
a person with a score of 6 to 30 points in the Korean-National Institutes of Health Stroke Scale (K-NIHSS) before endovascular recanalization therapy (ERT)
a person confirmed to have no disability before the onset of a disease because all tasks and daily activities can be performed before the onset of the disease by asking the person detailed questions about his or her conditions

Cohorts 1 and 2
a person with modified thrombolysis in cerebral infarction (mTICI) 2b or 3 grade reperfusion within 6 hours after the onset of the symptom (however, when angiography is performed for thrombectomy after tPA intravenous treatment, a person already confirmed to have mTICI2b-3 reperfusion before thrombectomy due to the venous tPA effect can also participate)
a person who can be administered a medicine for a clinical test within 6.5 hours after the onset of the symptom
a person who can be administered a medicine for a clinical test within 30 minutes after reperfusion of blood vessels
a person who can be subjected to MRI evaluation within 90 minutes after reperfusion of blood vessels
a person who voluntarily consents to participation in a clinical test in writing by himself/herself or a representative

Cohort 3
a person with modified thrombolysis in cerebral infarction (mTICI) 2b or 3 grade reperfusion within 10 hours after the onset of the symptom (however, when angiography is performed for thrombectomy after tPA intravenous treatment, a person already confirmed to have mTICI2b-3 reperfusion before thrombectomy due to the venous tPA effect can also participate)
a person who can be administered a medicine for a clinical test within 12 hours after the onset of the symptom
a person who can be administered a medicine for a clinical test within 2 hours after reperfusion of blood vessels
a person who can be subjected to MRI evaluation within 2 hours after reperfusion of blood vessels until administration of a medicine for a clinical test
a person with a pre-mRS of 0 to 1 before the onset of the disease
a person who voluntarily consents to participation in a clinical test in writing by himself/herself or a representative

Test subjects could not be registered in this clinical test when they met any one of the following criteria.
a person who has contraindications for endovascular recanalization therapy (as a result of an experimental result test, a person with a platelet value less than 40×109/L, a person with an aPTT of 50 seconds or more, or a person with an INR of more than 3.0)
a person who has a hypersensitivity reaction to a contrast medium or a medicine or component for a clinical test
a person who is contraindicated or unable to have an MRI examination
a person with a history of being predisposed to bleeding
a person with a history of hemorrhagic stroke within 6 months before participating in the clinical test
a person with a chronic liver disorder
renal disorder (serum creatine>3 mg/dL)
a person with a life expectancy of less than 3 months due to associated comorbidities other than stroke
a pregnant or lactating woman
a person who is administered tirofiban (anticoagulant) during endovascular recanalization therapy
a person who is administered/receives other medicines for a clinical test or medical devices within 12 weeks before screening
a person who is determined to be impossible to follow up
others who cannot participate in the clinical test at the discretion of the examiner

The following Cohorts 1 to 3 are administered using an infusion pump after mixing a test drug or control drug with an infusion solution.

Cohort 1. Low-Dose Administration Group
Test drug group: administration of the test drug is started within 30 minutes after reperfusion is confirmed within 6 hours after the onset of the symptom. Immediately after intravenous administration (bolus) of 150 mg of the test drug for 15 minutes, 750 mg of the test drug is intravenously IV infused for 23 hours and 45 minutes (f2 hours). Administration of the test drug should be started within 6.5 hours after the onset of the symptom.
Control drug group: a placebo is administered in the same manner as in the test drug group.

Cohort 2. High-Dose Administration Group
Test drug group: administration of the test drug is started within 30 minutes after reperfusion is confirmed within 6 hours after the onset of the symptom. Immediately after intravenous administration (bolus) of 300 mg of the test drug for 30 minutes, 1500 mg of the test drug is intravenously IV infused for 23 hours and 30 minutes (±2 hours). Administration of the test drug should be started within 6.5 hours after the onset of the symptom.
Control drug group: a placebo is administered in the same manner as in the test drug group.

Cohort 3. Low-Dose Administration Group
Test drug group: administration of the test drug is started within 2 hours after reperfusion is confirmed within 10 hours after the onset of the symptom. Immediately after intravenous administration (bolus) of 150 mg of the test drug for 15 minutes, 750 mg of the test drug is intravenously IV infused for 23 hours and 45 minutes (f2 hours). Administration of the test drug should be started within 12 hours after the onset of the symptom.
Control drug group: a placebo is administered in the same manner as in the test drug group.

Evaluation of Rate of Change in Cerebral Infarction Lesion

Diffusion weighted imaging (DWI) and gradient-recalled echo (GRE) or susceptibility weighted imaging (SWI), and magnetic resonance imaging (MRI) examination are performed within 90 minutes after reperfusion while administering a medicine for a clinical test, and this is utilized as a baseline result. A change ratio of cerebral infarction lesion at a time point of day 4 compared to the baseline was measured.

For comparison between a test group and the placebo, a two sample t-test is used after log conversion of an evaluation variable (infarct growth ratio). Additionally, a comparison between the test group and the placebo is made using a Wilcoxon rank sum test for a source data log which is not log-converted.

When factors that may affect prognosis should be corrected, analysis of covariance (ANCOVA) is used after log conversion of the evaluation variables (infarct growth ratio).

Evaluation

In patients who were administered a control drug or a test drug, the change ratio of cerebral infarction lesions among patients with similar baselines of cerebral infarction lesions was confirmed (Table 3).

TABLE 3
Change ratio of cerebral infarction lesion in patient who is administered clinical test drag

| | | | Cerebral infarction lesion | | | |
|---|---|---|---|---|---|---|
| | | | (cc) | | | Rate of increase |
| | | | | Time | Change ratio of | in cerebral |
| | | Clinical | | point of | cerebral | infarction lesion |
| Classification | Patient | test drug | Baseline | day 4 | infarction lesion | (%) |
| Cohort 1 | 1 | Control drug | 0.75 | 5.3 | 7.07 | 606.7 |
| | 2 | Test drug | 1.59 | 2.67 | 1.68 | 67.9 |
| | 3 | Control drug | 14.72 | 22.43 | 1.52 | 52.4 |
| | 4 | Test drug | 10.1 | 10.94 | 1.08 | 8.3 |
| | 5 | Control drug | 37.61 | 75.86 | 2.02 | 101.7 |
| | 6 | Test drug | 32 | 45.33 | 1.42 | 41.7 |
| | 7 | Control drug | 122.16 | 225.22 | 1.84 | 84.4 |
| | 8 | Test drug | 126.66 | 127.61 | 1.01 | 0.8 |

As a result of confirmation, it was shown that the group of patients who were administered the test drug had a significantly low rate of increase in cerebral infarction lesions compared to the group of patients who were administered the control drug.

Evaluation of Korean Version of Modified Rankin Scale (K-mRS)

The most widely used method for evaluating disability or dependence in daily life for people who have suffered from stroke or other neurological disorders is the Korean version of a modified Rankin Scale (K-mRS). The K-mRS is a global functional outcome scale which evaluates disability due to stroke and is the most universally used scale in stroke clinical tests. The K-mRS distribution shift analysis is a method of comparing the overall prognosis of the test group and the control group, and is the most recommended analysis method in clinical tests for acute stroke treatment methods including neuroprotective agents. The K-mRS is a scale of evaluating the patient's global functionality according to the independence of daily life and the degree to which the patient needs others' help, and is divided into 0 to 6 phases (0: normal, 5: severe disability, and 6: death). At the time points of days 29 and 90, a proportion of the K-mRS score evaluated by the evaluator is presented, and the pre-mRS evaluated at the time of screening is used as basic disease information. The proportions of K-mRS 0 to 2 (a state in which independent daily life is possible) and K-mRS 3 to 4 (a state in which all daily activities before the onset of stroke can be performed because there is no neurological disorder or there is a minor neurological disorder) are compared, and when patients showing poor prognosis are compared, the K-mRS 6 proportion traditionally corresponding to death is compared. However, since K-mRS 5 is as poor a prognosis as death, the present study also attempts to evaluate the K-mRS 5 to 6 proportions.

Analysis Method

Data on efficacy is analyzed using a modified intention-to-treat (mITT) and a Per-Protocol set (PPS). The main analysis target group at the time of efficacy evaluation is set as mITT. Data on safety is analyzed for the Safety set. The mITT includes data obtained from all test subjects who were administered a medicine for a clinical test after randomized allocation and had one or more primary efficacy evaluation results in the analysis. In addition, analysis is performed according to a randomly allocated group regardless of the medicine for a clinical test actually administered at the time of analysis.

Evaluation

The results of reviewing the efficacy from patients in Cohorts 1 and 2 are shown in the following Tables 4 and 5.

TABLE 4
K-mRS (continuous variable)
at the time point of day 90
K-mRS (continuous variable) at the time point of day 90

| Cohort 1 | Test drug (N = 9) | Control drug (N = 5) |
|---|---|---|
| Mean(SD) | 1.44(1.42) | 2.60(2.07) |
| Median | 1.00 | 3.00 |
| 1st Quartile | 0.00 | 1.00 |
| 3rd Quartile | 2.00 | 4.00 |
| Min, Max | 0.00, 4.00 | 0.00, 5.00 |
| P-value [1] | — | 0.3081 |

K-mRS classification at the time point of day 90, n(%)

| | | |
|---|---|---|
| K-mRS 0 | 3(33.33) | 1(20.00) |
| K-mRS 1 | 2(22.22) | 1(20.00) |
| K-mRS 2 | 2(22.22) | 0 |
| K-mRS 3 | 1(11.11) | 1(20.00) |
| K-mRS 4 | 1(11.11) | 1(20.00) |
| K-mRS 5 | 0 | 1(20.00) |
| K-mRS 6 | 0 | 0 |
| P-value [2] | — | 0.6273 |

TABLE 5
K-mRS classification at the time point of day 90
K-mRS classification at the time point of day 90, n(%)

| | Cohort 1 | | Cohort 2 | |
|---|---|---|---|---|
| | Test | Control | Test | Control |
| | drug | drug | drug | drug |
| | (n = 9) | (n = 5) | (n = 6) | (n = 5) |
| K-mRS 0~2 | 7(77.78) | 2(40.00) | 1(20.00) | 2(40.00) |
| K-mRS 3~4 | 2(22.22) | 2(40.00) | 3(60.00) | 3(60.00) |
| K-mRS 5~6 | 0 | 1(20.00) | 1(20.00) | 0 |
| P-value [1] | — | 0.3287(f) | — | 1.0000(f) |

As a result of examining efficacy, it was confirmed that in the drug to which 900 mg of the test drug was administered, the results of the K-mRS of the patient group to which the test drug was administered were far superior to those of the patient group to which the control drug was administered (Table 5).

1.-21. (canceled)

22. A method for treating stroke, comprising administering, to a subject in need thereof, a pharmaceutic composition comprising a therapeutically effective amount of 10-ethoxy-8-(morpholinomethyl)-1,2,3,4-tetrahydrobenzo[h][1,6]naphthyridin-5(6H)-one, a pharmaceutically acceptable salt thereof, a hydrate thereof, a salt hydrate thereof or a solvate thereof as an active ingredient and a pharmaceutically acceptable carrier, wherein the pharmaceutical composition is divided into a first dose of the pharmaceutical composition and a second dose of the pharmaceutical composition and administered, a first dose of the pharmaceutical composition comprising 8 to 20 wt % of the active ingredient based on a single dose of the active ingredient is intravenously administered to a subject at 5 to 15 mg/min based on the active ingredient, and a second dose of the pharmaceutical composition comprising the remaining dose of the active ingredient is intravenously administered to the subject for 20 to 26 hours.

23. The method of claim 22, wherein the single dose of the active ingredient is 700 to 2000 mg.

24. The method of claim 22, wherein the single dose of the active ingredient is 700 to 1100 mg.

25. The method of claim 24, wherein the first dose of the pharmaceutical composition is intravenously administered to the subject at 7 to 13 mg/min based on the active ingredient, and the second dose of the pharmaceutical composition is intravenously administered to the subject for 20 to 26 hours.

26. The method of claim 22, wherein the first dose of the pharmaceutical composition and the second dose of the pharmaceutical composition are sequentially and continuously administered to the subject.

27. The method of claim 22, wherein the pharmaceutical composition is administered to a subject who requires reperfusion due to the onset of a stroke symptom or a subject who has undergone reperfusion after the onset of a stroke symptom.

28. The method of claim 22, wherein the pharmaceutical composition is administered within 12 hours after the onset of a stroke symptom.

29. The method of claim 27, wherein the reperfusion is performed within 10 hours after the onset of a stroke symptom.

30. The method of claim 22, wherein a blood concentration of the active ingredient after administration of the pharmaceutical composition is 1000 μg/L or more for 24 hours.

31. The method of claim 22, wherein the pharmaceutical composition is administered in combination with tPA.

32. The method of claim 22, wherein the subject is a mammal.

33. The method of claim 32, wherein the subject is a human.

34. The method of claim 32, wherein a Korean version of a modified Rankin Scale (K-mRS) measured at a time point of day 90 after administration of the pharmaceutical composition to the subject is 2.5 or less.

35. The method of claim 22, wherein the pharmaceutical composition further comprises a stabilizer and/or a pH adjuster.

36. The method of claim 35, wherein the pH adjuster is sodium hydrogen carbonate, sodium hydroxide, sodium citrate, sodium phosphate, potassium hydroxide, potassium carbonate, potassium phosphate or a mixture thereof.

37. The method of claim 35, wherein the stabilizer is a sugar or a derivative thereof.

38. The method of claim 35, wherein a pH of the pharmaceutical composition 2.5 to 7.
